(19) United States
(12) Patent Application Publication
(10) Pub. No.: US 2009/0139524 A1
(43) Pub. Date: Jun. 4, 2009

(54) OXYGENATING DEVICE AND METHOD

(75) Inventor: Philip Stuart Esnouf, Hawthorn (AU)

Correspondence Address: SEED INTELLECTUAL PROPERTY LAW GROUP PLLC, 701 FIFTH AVE, SUITE 5400, SEATTLE, WA, 98104, US

(73) Assignee: Ultimate Medical Pty. Ltd.

(21) Appl. No.: 12/065,496

(22) PCT Filed: Aug. 30, 2006
(86) PCT No.: PCT/AU2006/001259

§ 371 Date: Oct. 20, 2008

(30) Foreign Application Priority Data
Aug. 30, 2005 (AU) 2005904739

(51) Int. Cl. A61M 16/08

(52) U.S. Cl. 128/205.13

(57) ABSTRACT

An oxygenating device including: a body having: a first coupling spigot to which a collapsible bag is connected; an oxygen inlet to which a supply of oxygen can be connected for inflating, in use, the collapsible bag with oxygen; a second coupling spigot having a female leur socket for receipt in use of a male leur connector of an endotracheal tube or laryngeal mask; and a mask which can be applied, in use, about the nose and mouth of a patient and having an inlet/outlet socket mounted over the second coupling spigot.

### BACKGROUND

1. Technical Field

This invention relates to an oxygenating device and method.

2. Description of the Related Art

Post-operative treatment of patients usually includes a number of hours spent in a recovery room where the patients can be monitored whilst the effects of anesthetic wear off. It is desirable to supply oxygen or oxygen enriched air to the patients in the recovery room to assist in returning to normal breathing and to offset the effects of the anesthetic. In most recovery rooms, oxygen is available from bedside outlets which are capable of delivering oxygen at a relatively low rate, say of the order of about 4 to 6 liters per minute. This is typically less than the oxygen requirements of a typical adult patient. Australian Patent No. 721704 discloses a disposable oxygenating device which stores oxygen from the low volume supply in a collapsible plastic bag and enables a relatively large volume of oxygen or oxygen rich air to be supplied to the patient from the bag during the patient's inspiration cycle.

When patients leave the operating theatre, it is customary to leave the laryngeal mask in the patient. The free end of the laryngeal mask is fitted with a male leur connector of standard size which can be connected to an anesthetic machine in the operating theatre. The known form of oxygenating device includes a female leur socket which can receive the male leur connector of the endotracheal tube or laryngeal mask which is still projecting from the mouth of the patient who has been moved from the operating theatre into the recovery room. The oxygen device increases the supply of oxygen to the patient in the post operative stage so as to assist in more quickly overcoming the effects of the anesthetic.

There are, however, circumstances where oxygenation is required in circumstances where an endotracheal tube or laryngeal mask is not fitted to a patient or the endotracheal tube or laryngeal mask has been removed.

### BRIEF SUMMARY

Accordingly, it is an object of the invention to provide an oxygenating device which can be used in conjunction with an endotracheal tube or laryngeal mask or alternatively when neither of those devices is fitted to a patient.

According to the present invention there is provided an oxygenating device including:

a body having:

a first coupling spigot to which a collapsible bag is connected;

an oxygen inlet to which a supply of oxygen can be connected for inflating, in use, the collapsible bag with oxygen;

a second coupling spigot having a female leur socket for receipt in use of a male leur connector of an endotracheal tube or laryngeal mask; and

a mask which can be applied, in use, about the nose and mouth of a patient and having an inlet/outlet socket mounted over said second coupling spigot.

Preferably, the mask is molded from plastics material and includes a sidewall or sidewalls having a free edge which, in use, engages the face of a patient, the sidewall or sidewalls being foldable into a non-operative position in which the free edge does not, in use, engage the face of the patient and oxygen or oxygen enriched air is supplied to the patient through the endotracheal tube or laryngeal mask.

When, however, the endotracheal tube or laryngeal mask is removed from the patient, the mask can be folded into an operative position in which its free edge engages the face of the patient. In this mode of use, oxygen or oxygen rich air is supplied to the interior or the mask, during an inspiration cycle of the patient, from oxygen or oxygen enriched air stored in the collapsible bag.

There are also circumstances where oxygenation of a patient is desirable where the procedure does not involve the use of an endotracheal tube or laryngeal mask. For instance, if anesthetic is administered to a patient intravenously or regionally, the device of the invention can be used simply by fitting the mask to the patient and supplying oxygen to the oxygen inlet.

It is also preferred that the device of the invention is lightweight and disposable. Preferably, components of the device are molded from plastics material to which the collapsible bag can be connected by heat welding or ultrasonic welding. It is desirable to have a relatively low weight for the device because it is mounted on the projecting end of the laryngeal mask or endotracheal tube. In this respect it is preferred that the weight of the device of the invention is in the range from 25 to 40 grams and preferably about 30 grams.

The invention also provides a method of oxygenating a patient including the steps of:

inserting a male leur connector of an endotracheal tube or laryngeal mask applied to a patient into a female leur socket of an oxygenating device, the device including a collapsible bag which is supplied with oxygen, the device also including a folded mask;

removing the endotracheal tube or laryngeal mask from the patient and the oxygenating device;

unfolding the mask;

fitting the mask about the nose and mouth of the patient; and

continuing to supply oxygen to the collapsible bag so that oxygenation of the patient continues.

The invention also provides a method of oxygenating a patient including the steps of:

inserting a male leur connector of an endotracheal tube or laryngeal mask applied to a patient into a female leur socket of an oxygenating device, the device including a collapsible bag which is supplied with oxygen;

removing the endotracheal tube or laryngeal mask from the patient and the oxygenating device;

fitting a mask about the nose and mouth of the patient;

connecting the same oxygenating device to the mask; and

continuing to supply oxygen to the collapsible bag so that oxygenation of the patient continues.

The invention also provides a kit for assembly into an oxygenating device, the kit including:

a body having:

a first coupling spigot to which a collapsible bag is connected;

an oxygen inlet to which a supply of oxygen can be connected for inflating, in use, the collapsible bag with oxygen;

a second coupling spigot having a female leur socket for receipt in use of a male leur connector of an endotracheal tube or laryngeal mask; and

a mask which can be applied, in use, about the nose and mouth of a patient and having an inlet/outlet socket which can, in use, be mounted over the second coupling spigot, whereby:

in a first mode of assembly of the kit, the male leur connector of an endotracheal tube or laryngeal mask is inserted into the second coupling spigot; and,

in a second mode of assembly of the kit, the inlet/outlet socket of the mask is mounted over said second coupling spigot.

### BRIEF DESCRIPTION OF THE SEVERAL VIEWS OF THE DRAWINGS

The invention will now be further described with reference to the accompanying drawings, in which:

FIG. 1 is a schematic cross-sectional side view of a known form of oxygenating device;

FIG. 2 is a cross-section along the line 2-2;

FIG. 3 is a schematic end view of the device shown in FIG. 1;

FIG. 4 is a side view of the oxygenating device of the invention;

FIG. 5 is a fragmentary frontal view of the device of the invention;

FIG. 6 is a schematic view which shows the mask in a non-operative position; and

FIG. 7 is a schematic view showing the mask folded into its operative position on a patient.

### DETAILED DESCRIPTION

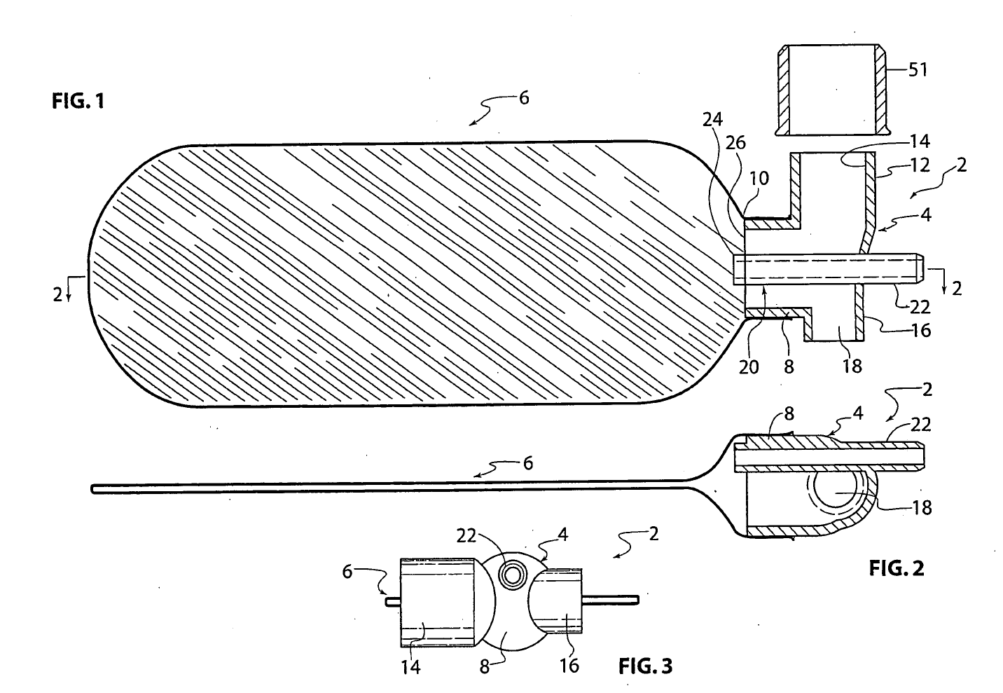

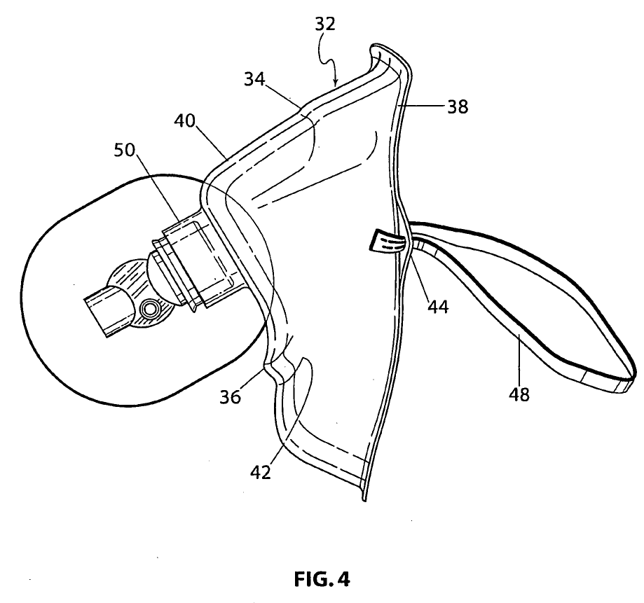

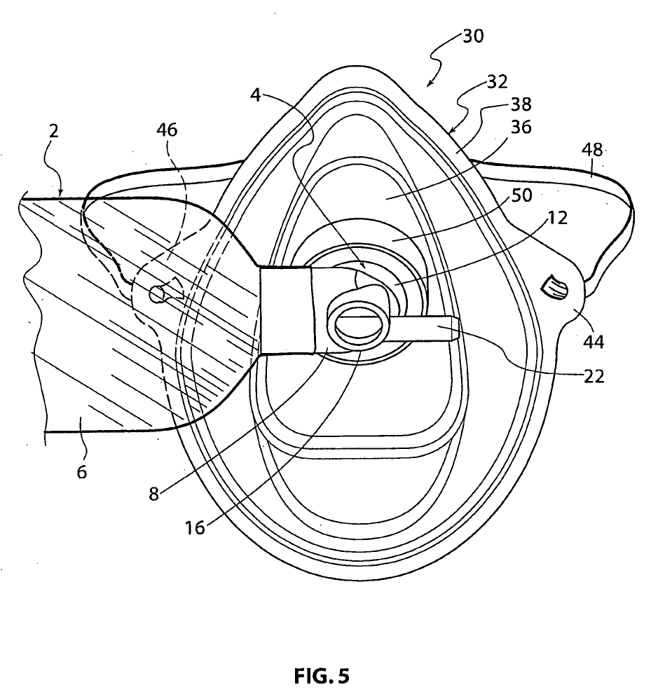

FIGS. 1 to 3 illustrate an oxygenating device 2 of known type such as devices of the type shown in Australian Patent No. 721704, the content of this specification being incorporated herein by cross reference. Devices of this type are known as T-Bags, supplied by Ultimate Medical Pty. Ltd. of Burnley, Victoria, Australia, which have been widely used in Europe and Australia.

Briefly, the device 2 includes a body 4 molded from plastics material and having a collapsible bag 6 of sheet plastics material connected thereto. The bag 6 is not resilient in the sense of being a resilient bladder-like body which is used in some types of resuscitation devices such as disclosed in U.S. Pat. No. 3,196,866. The body 4 is essentially hollow and includes a first spigot 8 to which a mouth 10 of the bag is connected by heat or ultrasonic welding. The body 4 includes a second coupling spigot 12, the interior socket of which is tapered so as to form a standard female leur socket 14. The body 4 includes an outlet spigot 16 which defines an outlet port 18 from which expiration products from the patient can pass, as will be described below. The body 4 includes an oxygen supply tube which is integrally molded with the body 4, the oxygen supply tube 20 extending generally through the body and having a projecting inlet spigot 22 to which a supply of oxygen can be connected. The tube 20 has an outlet orifice 24 which projects somewhat beyond the end face 26 of the first spigot 8 so that oxygen from the tube 20 is directed towards the interior of the bag 6.

In use, a male leur connector (not shown) is connected to the leur socket 14 and an oxygen supply line (not shown) is connected to the oxygen inlet spigot 22. Oxygen inflates the bag 6 so that when the patient breathes in, oxygen accumulated in the inflated bag 6 is sucked into the endotracheal tube or laryngeal mask so as to supply oxygen or oxygen rich air to the patient. During the expiration cycle of the patient, expiration products tend to pass directly from the socket 14 to the outlet port 18 which is located opposite thereto so that relatively little of the expired gases will enter into the bag 6. In this way oxygen or oxygen enriched air is available to a patient even though the oxygen supply line coupled to the oxygen inlet spigot is capable of only low flow rates.

Devices of the type shown in FIGS. 1 to 3 are widely used in many recovery rooms because they are a cheap and effective way of supplying oxygen to post-operative patients. The devices also have the advantage that the breathing of a patient can be quickly monitored by visual observation of the movement of the bag caused by inspiration and expiration of the patient. Movement of the bag gives an indication of the depth and rate of breathing of the recovering patient. Where a number of patients are in the recovery room, it is relatively easy for medical staff to observe patients having low breathing depth and/or rate and so attention can be given to those patients.

As described above, the known type of device is used in conjunction with an endotracheal tube or laryngeal mask applied to a patient. There are, however, circumstances in which a disposable oxygenating device is required where an endotracheal tube or laryngeal mask has not been used or has been removed from a patient.

FIGS. 4 to 7 illustrate an oxygenating device 30 constructed in accordance with the invention. The device 30 includes a mask 32 which is coupled to an oxygenating device 2 of the same type as described above. The mask 32 can be of known type, for instance of the type supplied by INTERSURGICAL known as EUROSTYLE 1104 ADULT STYLE. This type of mask has ventilation holes in the sidewall and when used in the device of the invention, these holes need to be covered by an adhesive patch or by other means so that the mask is essentially impervious. This type of mask is molded from transparent flexible plastic material such as polyvinylchlorine and typically has a wall thickness of about 1 mm. The mask 32 includes a sidewall 34, front face 36 and a rearward peripheral flange 38. The sidewall 34 includes an upper portion 40 which in use is located adjacent to the nose of a patient and a lower portion 42 which, in use, is located near the mouth of a patient. The peripheral flange 38 includes lateral tabs 44 and 46 having holes therein for fixing ends of an elastic strap 48. In use of the mask 32, the flange 38 engages the face of the patient so that the mask generally envelops the nose and mouth of the patient. The mask 32 includes an integral spigot 50 which is normally coupled to a supply fitting (not shown) for supply of oxygen or the like. In accordance with the invention, however, the second coupling spigot 12 of the device 2 is inserted in the spigot 50 of the mask. A sleeve 51 (as shown in FIG. 1) may be provided in order to ensure a neat fit of the spigot 12 within the spigot 50 of the mask. Alternatively, the diameter of the spigot 50 could be molded with an internal diameter such that the spigot 12 can be directly inserted in the spigot 50. The spigot 50 could be bonded to the spigot 12 or alternatively would be detachable therefrom. The sidewall 34 does not include any ventilation holes, as in the INTERSURGICAL mask referred to above. In this arrangement because the flange 38 engages the face of the patient, substantially the only gas available to the patient during his or her inspiration cycle is via the bag 6. The mask 32 may, however, include a sampling port (not shown) for sampling gas from within the mask. The diameter of the sampling port is preferably in the range from 1.5 mm to 2.5 mm. It would be possible to cover the port by means of an adhesive patch (not shown). Alternatively, the port could be left open but the amount of gas admitted through such a small port would be relatively negligible compared to the gas which is made available to the patient through the bag 6, during the inspiration cycle.

The device of the invention can be supplied in a condition in which the oxygenating device 2 is connected to the spigot of the mask 32. This could be a permanent connection or alternatively could be a detachable connection.

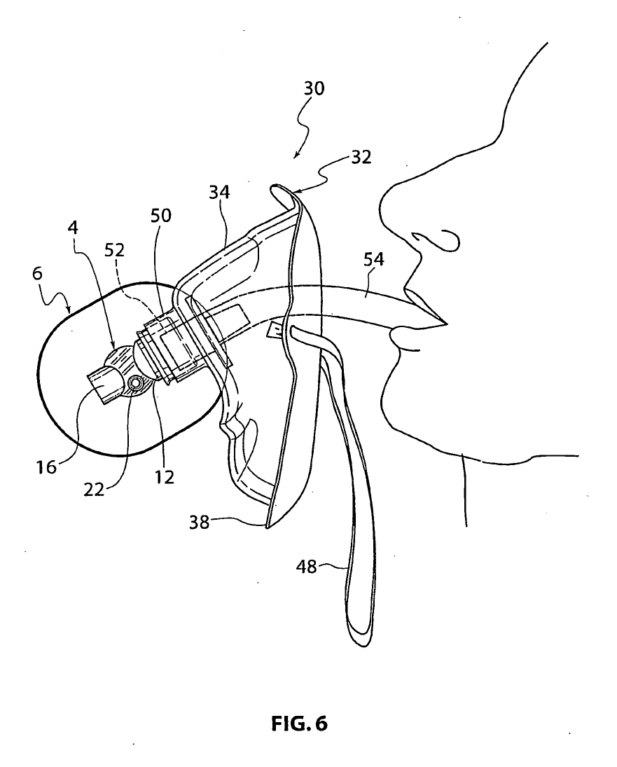

If the oxygenating device 2 is detachably connected to the mask, then it is possible to initially connect the oxygenating device 2 to a laryngeal mask 54 (as shown in FIG. 6) and, after removal of the laryngeal mask 54 from the patient, the face mask 32 can be applied to the patient and the same T-bag can be connected to the spigot 50 of the mask 32.

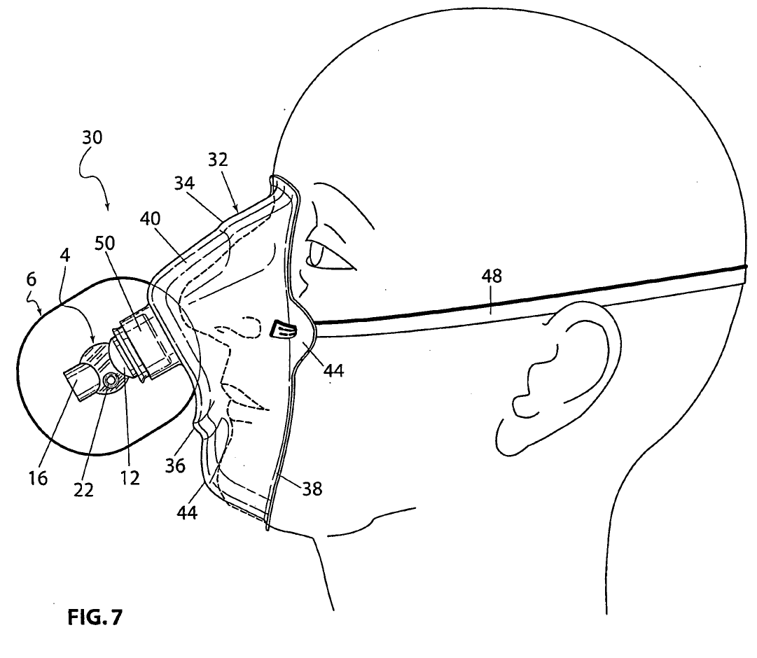

FIGS. 6 and 7 illustrate an alternative mode of use of the device of the invention in which the sidewall 34 of the mask is folded initially into a non-operative position, as diagrammatically illustrated in FIG. 6. Where the mask is molded especially for use in the oxygenating device of the invention, it can be molded with fold lines (not shown) in the sidewall 34 thereof so as to define the way in which folding takes place.

In the non-operative position, the peripheral flange 38 is folded forwardly so that it lies considerably forwardly of its position shown in FIG. 4. The flange 38 may be roughly in a plane which includes the spigot 50. The resilience of the sidewall 34 is such that it is in a stable condition in its non-operative position. In this mode of use, a male leur connector 52 of an endotracheal tube or laryngeal mask 54 can be inserted directly into the female leur socket 14 of the body 4. As the leur connector 52 is normally projecting some distance from the mouth of the patient, the folded mask 32 is clear of the face of the patient and all breathing of the patient takes place through the endotracheal tube or laryngeal mask 54.

When, however, the endotracheal tube or laryngeal mask 54 is removed from the patient, the sidewalls 34 of the mask can be unfolded to the operative position as shown in FIG. 7 and breathing of the patient takes place through the mouth and/or nose, and oxygen or oxygen rich air is supplied to the interior of the mask 32 via the device 2. This avoids the need for two oxygenating devices 2 to be used during oxygenation with the endotracheal tube or laryngeal mask present and oxygenation after removal of the endotracheal tube or laryngeal mask. Some times patients will remove a laryngeal mask themselves (without the permission of medical staff) and if this is done, again the same device 2 can be used, avoiding the need for use of a fresh oxygenating device 2.

The device of the invention is molded from inexpensive lightweight materials and is therefore disposable. It does not have any valves and this makes its construction simpler and lightweight, the weight of the device typically being in the range from 25 to 40 grams and preferably about 30 grams.

The oxygenating device of the invention can be supplied in the form of a kit. Preferably, the kit includes an oxygenating device 2, mask 32 and a length of oxygen tubing (not shown). The oxygen tubing may be the standard form of tubing which is used for oxygen supply. Typically a length would be about 3 meters and having elastic sockets either end thereof so that the tube can be fitted to various oxygen supplies at one end and at the other to the inlet spigot 22. Normally the tube is of PVC having a plasticizer therein and typically having an outer diameter of say 6 mm and an internal diameter of say 4 mm. The internal bore of the tube is preferably internally ribbed so as to prevent inadvertent closing off of the tube in the event that it is kinked. The various components can be packed in a transparent plastic bag (not shown) and supplied to the surgical staff who can then assemble the components of the kit in the appropriate manner.

The reference to any prior art in this specification is not, and should not be taken as, an acknowledgment or any form of suggestion that that prior art forms part of the common general knowledge in Australia.

Many modifications will be apparent to those skilled in the art without departing from the spirit and scope of the invention.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An oxygenating device including:
a body having:
a first coupling spigot to which a collapsible bag is connected;
an oxygen inlet to which a supply of oxygen can be connected for inflating, in use, the collapsible bag with oxygen;
a second coupling spigot having a female leur socket for receipt in use of a male leur connector of an endotracheal tube or laryngeal mask; and
a mask which can be applied, in use, about a nose and a mouth of a Face of a patient, the mask having an inlet/outlet socket mounted over said second coupling spigot.

2. A device as claimed in claim 1 wherein the mask includes a peripheral flange which in use engages or lies closely adjacent to the face of the patient so that substantially all of the gas available to the patient during an inspiration cycle is delivered from said bag.

3. A device as claimed in claim 1 wherein the mask is moulded from flexible plastics material and including at least one sidewall having a free edge which in use engages the face of a patient, the at least one sidewall being foldable into a non-operative position in which the free edge does not, in use, engage the face of the patient and oxygen or oxygen rich air is supplied to the patient through the endotracheal tube or laryngeal mask.

4. A device as claimed in claim 3 wherein a hollow adaptor sleeve is located on said second coupling spigot and the adaptor sleeve is inserted in the inlet/outlet socket of the mask.

5. A device as claimed in claim 4 wherein the mask is detachably mounted to the second coupling spigot or adaptor sleeve.

6. A device as claimed in claim 3 wherein the mask is bonded or welded to the second coupling spigot or adaptor sleeve.

7. A device as claimed in claim 1 wherein the mask is transparent.

8. A device as claimed in claim 1 wherein the mask is substantially imperforate.

9. A device as claimed in 1 wherein the mask includes a sampling port.

10. A device as claimed in claim 9 wherein a diameter of the sampling port is in the range 1.5 mm to 2.5 mm.

11. A method of oxygenating patients, the method comprising:
inserting a male leur connector of an endotracheal tube or laryngeal mask applied to a patient into a female leur socket of an oxygenating device, the device including a collapsible bag which is supplied with oxygen, the device also including a folded mask;
removing the endotracheal tube or laryngeal mask from the patient and the oxygenating device;
unfolding the mask;
fitting the mask about a nose and a mouth of the patient; and
continuing to supply oxygen to the collapsible bag so that oxygenation of the patient continues.

12. A method as claimed in claim 11 further comprising:
folding a free edge of the mask into a non-operative position in which the free edge does not, in use, engage a face of the patient.

13. A method of oxygenating a patient, the method comprising:
inserting a male leur connector of an endotracheal tube or laryngeal mask applied to a patient into a female leur socket of an oxygenating device, the device including a collapsible bag which is supplied with oxygen;
removing the endotracheal tube or laryngeal mask from the patient and the oxygenating device;
fitting a mask about a nose and a mouth of the patient;
connecting the same oxygenating device to the mask; and
continuing to supply oxygen to the collapsible bag so that oxygenation of the patient continues.

14. A kit for assembly into an oxygenating device, the kit including:
a body having:
a first coupling spigot to which a collapsible bag is connected;
an oxygen inlet to which a supply of oxygen can be connected for inflating, in use, the collapsible bag with oxygen;
a second coupling spigot having a female leur socket for receipt in use of a male leur connector of an endotracheal tube or laryngeal mask; and
a mask which can be applied, in use, about a nose and a mouth of a patient and having an inlet/outlet socket which can, in use, be mounted over the second coupling spigot, whereby:
in a first mode of assembly of the kit, the male leur connector of an endotracheal tube or laryngeal mask is inserted into the second coupling spigot; and,
in a second mode of assembly of the kit, the inlet/outlet socket of the mask is mounted over said second coupling spigot.

15. A kit as claimed in claim 14 further comprising a length of tube, one end of which can be coupled to said supply of oxygen and the other end of which can be coupled to said oxygen inlet.

16. A kit as claimed in claim 15 wherein the length of tube is about 3 meters long.

17. A kit as claimed in claim 16 wherein the length of tube has elastic sockets on each end thereof.

18. A kit as claimed in claim 17 wherein the body, the mask and the length of tube are packed within a bag.

19. A kit as claimed in claim 18 wherein the bag is a transparent plastic bag.

20. A kit as claimed in claim 15 wherein the length of tube has elastic sockets in each end thereof and the body, the mask and the length of tube are packed within a bag.
